(19) United States
(12) Patent Application Publication
(10) Pub. No.: US 2023/0168233 A1
(43) Pub. Date: Jun. 1, 2023

(54) Portable Smart Air Quality Multisensory System Equipped Carrying Case for Asthma Inhalers

(71) Applicants: Satyanarayan Hegde, Gainesville, FL (US); University of Florida Research Foundation, Inc., Gainesville, FL (US); University of North Carolina at Chapel Hill, Chapel Hill, NC (US)

(72) Inventors: RAVI S. SRINIVASAN, Gainesville, FL (US); S M SHAHRIAR NIRJON, Chapel Hill, NC (US); SHERRY AHRENTZEN, Gainesville, FL (US); SATYANARAYAN HEGDE, Gainesville, FL (US)

(21) Appl. No.: 18/154,588

(22) Filed: Jan. 13, 2023

Related U.S. Application Data
(63) Continuation of application No. PCT/US2021/041757, filed on Jul. 15, 2021.
(60) Provisional application No. 63/052,206, filed on Jul. 15, 2020.

(51) Int. Cl. G01N 33/00; A61M 15/00; G16H 20/13; G16H 40/67

(52) U.S. Cl. CPC G01N 33/0075; A61M 15/0001; G16H 20/13; G16H 40/67; A61M 2230/20; A61M 2230/42

(57) ABSTRACT

The present disclosure presents systems, apparatuses, and methods of evaluating air quality sensor data. In this regard, a method comprises receiving air quality sensor data from a plurality of portable smart air quality measurement system (SAQMS) communication devices from a plurality of citizens in a geographic location; receiving air quality sensor data from an air quality measurement and calibration (AQMC) station in the geographic location; determining a level of resolution for one or more sensors of a portable SAQMS communication device at a central evaluation and measurement service; correcting air quality data received from the portable SAQMS communication device to compensate for the determined level of resolution at the central evaluation and measurement service; and generating a map of air quality data based on at least the corrected air quality data of the portable SAQMS communication device and a plurality of other portable SAQMS communication devices.

### CROSS-REFERENCE TO RELATED APPLICATION

This application claims priority to and is a continuation of PCT Application No. PCT/US2021/041757, filed on Jul. 15, 2021, which claims priority to U.S. provisional application entitled, “Portable Smart Air Quality Multisensory System Equipped Carrying Case for Asthma Inhalers,” having serial number 63/052,206, filed Jul. 15, 2020, each of which is entirely incorporated herein by reference.

### TECHNICAL FIELD

The present disclosure is generally related to air quality sensors.

### BACKGROUND

Asthma is a chronic disease that often causes exacerbation of disease activity, some of which result in hospitalizations. Asthmatic children spend 60% of their waking hours in school. A recent large-scale study showed that co-exposure to elevated endotoxin levels and fine particulate matter (e.g., PM_{2.5}) was synergistically associated with increased emergency room visits for asthma among children. Air quality measures such as PM_{2.5}, NO₂, and O₃, and dampness-related contaminants play a significant role in asthma exacerbation as well as disease progression. Currently, only a limited number of air monitoring station exists to cover any general geographic area, making it impossible to identify ground-level O₃, PM_{2.5}, and other air pollutant levels that may exceed National Ambient Air Quality Standards (NAAQS) in specific neighborhoods or community pockets.

### SUMMARY

Embodiments of the present disclosure provide systems and methods for evaluating air quality sensor data. Briefly described, one embodiment of the method comprises receiving air quality sensor data from a plurality of portable smart air quality measurement system (SAQMS) communication devices in a form of an asthma inhaler carrying case from a plurality of citizens in a geographic location; receiving air quality sensor data from an air quality measurement and calibration (AQMC) station in the geographic location; determining a level of resolution for one or more sensors of a portable SAQMS communication device at a central evaluation and measurement service; correcting air quality data received from the portable SAQMS communication device to compensate for the determined level of inaccuracy at the central evaluation and measurement service; and generating a map of air quality data based on at least the corrected air quality data of the portable SAQMS communication device and a plurality of other portable SAQMS communication devices.

Briefly described, one embodiment of the system, among others, can include a carrying case having an interior pouch; a spirometer device integrated with the carrying case, wherein the spirometer device has a pressure sensor and a spirometer tube coupled to the pressure sensor, wherein the spirometer devices a mouthpiece that is configured to be extended away from a body of the carrying case; a plurality of air quality sensors integrated with the carrying case, the plurality of air quality sensors including sensors for measuring O₃, PM_{2.5}, NO₂, temperature, and relative humidity levels; a global positioning system (GPS) sensor integrated with the carrying case and configured to provide location data for the carrying case; and hardware circuitry configured to transmit sensor data from at least the pressure sensor, air quality sensors, or GPS sensor to a base station.

In one or more aspects for such systems and/or methods, an exemplary system/method can further perform operations comprising extending a mouthpiece of the spirometer device away from a body of the portable SAQMS communication device; attaching the portable SAQMS communication device to an asthma inhaler; publishing the generated map for public consumption; receiving usage data from the plurality of portable SAQMS communication devices, wherein the usage data indicates a volume of air inspired and expired by the lungs of a plurality of users during use of asthma inhaler devices; receiving usage data from the plurality of portable SAQMS communication devices, wherein the usage data indicates occurrences when respective asthma inhaler devices are used by a plurality of users; and/or receiving breathing sound data from the plurality of portable SAQMS communication devices, wherein the breathing sound data are recorded during usage of a spirometer device or an asthma inhaler that is coupled or attached to a respective portable SAQMS communication device.

In one or more aspects for such systems and/or methods, the portable SAQMS communication device is integrated with a spirometer device; the spirometer device has a pressure sensor and a spirometer tube coupled to the pressure sensor; the portable SAQMS communication device is integrated with a pulse oximeter device; the pulse oximeter device is located in an inner tube of the spirometer device; the portable SAQMS communication device comprises a plurality of air quality sensors, the plurality of air quality sensors including sensors for measuring O₃, PM_{2.5}, NO₂, temperature, and relative humidity levels; the portable SAQMS communication device communicates with one or more servers for the central evaluation and measurement service using cellular communications; the portable SAQMS communication device communicates with one or more servers for the central evaluation and measurement service using WiFi communication; the portable SAQMS communication device communicates with the air quality measurement and calibration (AQMC) station using WiFi communications; the portable SAQMS communication device communicates with the air quality measurement and calibration (AQMC) station using short range communications; the generated map indicates an air quality level inside a building depicted in the generated map; the generated map identifies regions having a high level of pollutants as well as safe zones; the air quality sensor data is provided with location data for the geographic location; and/or the sensor data transmitted by hardware circuitry further comprises breathing sounds recorded via the one or more microphones or data obtained by the pulse oximeter device.

Other systems, methods, features, and advantages of the present disclosure will be or become apparent to one with skill in the art upon examination of the following drawings and detailed description. It is intended that all such additional systems, methods, features, and advantages be included within this description and be within the scope of the present disclosure.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a block diagram of an exemplary air quality measurement and communication system in accordance with various embodiments of the present disclosure.

FIG. 2 shows a computer-generated sketch of an exemplary case of a portable Smart Air Quality Multisensory System (SAQMS) communication device in conjunction with an asthma inhaler device in accordance with various embodiments of the present disclosure.

FIG. 3-4A show computer-generated drawings of an exemplary case of a portable Smart Air Quality Multisensory System (SAQMS) communication device that correspond to FIG. 2 with an asthma inhaler device inserted in the case (FIG. 3) and with the asthma inhaler device, spirometer device, and pulse-oximeter not inserted in the case (FIG. 4A), in accordance with various embodiments of the present disclosure.

FIG. 4B shows a prototype image of an exemplary case of the portable SAQMS communication device that corresponds to FIG. 2 with an asthma inhaler device inserted in the case.

FIG. 5 depicts varying scales or levels of an exemplary air quality map being available for viewing, such a building, campus, city, and state scales/levels of the map, in accordance with various embodiments of the present disclosure.

FIG. 6 shows an exemplary process flow for evaluating and mapping air quality sensor data in accordance with various embodiments of the present disclosure.

FIG. 7 depicts a schematic block diagram of a computing device that can be used to implement various embodiments of the present disclosure.

### DETAILED DESCRIPTION

Per the United States National Institute of Health (NIH), 15 million Americans have asthma including 5 million children. With higher pollution and other activities, this number is expected to increase significantly. Among others, one of the ways to manage asthmatic conditions is the use of inhalers. When conditions arise (e.g. shortness of breath, etc.), a subject uses an inhaler to dilate the airways in the lungs of the subject. Some common triggers of asthma are environmental pollutants (O₃, PM_{2.5}, PM₁₀, NO₂, etc.) and relative humidity - both inside and outside buildings. The present disclosure describes various embodiments of systems, apparatuses, and methods of monitoring, acquiring, and/or delivering air quality measurements using a portable air quality measurement (PAQM) communication device, also referred to as a portable Smart Air Quality Multisensory System (SAQMS) equipped carrying case, such as may be used for carrying asthma inhalers.

An exemplary SAQMS communication device, in accordance with various embodiments of the present disclosure, can attach to a subject’s inhaler device, and be configured to facilitate the providing of real-time, high-resolution, spatial and temporally-scaled air quality data in connection with a centralized evaluation and mapping service. Correspondingly, an exemplary centralized evaluation and mapping service of the present disclosure is unique in that a central server/platform can monitor real-time air quality data gathered by individual measurement & communication devices, which can then collectively be used to create real-time air quality data. While the United States Environmental Protection Agency’s (EPA) does have select air quality monitoring stations, these stations are not generally available away from a city locale and may have limited types of air quality sensors. The availability of real-time, high-resolution, calibrated, air quality data can be advantageous for a community in general, including at-risk individuals, and also for companies & organizations that offer products or services that are directed to, relied upon, or affected by air quality metrics. For example, using real-time, high-resolution air quality data, communities can make informed choices for prioritizing building interventions in light of the elderly’s and children’s respiratory health as well as cost-effectiveness and other concerns. Among all other population, the elderly and children are more vulnerable to air pollutants. As a non-limiting example, such information can effectively identify environmental pollutants that can exacerbate respiratory conditions and direct strategies for building filtration system modifications that can reduce asthma triggers. With real-time, high resolution calibrated environmental data, local, state, and national policies can be implemented for effective air quality management.

In various embodiments, an exemplary portable SAQMS communication device is adapted to include a plurality of air quality sensors, such as, but not limited to, sensors for measuring O₃, PM_{2.5}, PM₁₀, NO₂, temperature, RH (relative humidity), and GPS (global positioning system) levels, values, or readings. Thus, the portable SAQMS communication device can act as a personal air quality sensor that follows an individual as the individual moves about his/her surroundings without having to rely on fixed air quality sensors (at monitoring stations) that are sparsely located in a user’s surroundings and may miss potentially threatening air quality scenarios that pose high health risk to individuals, including children.

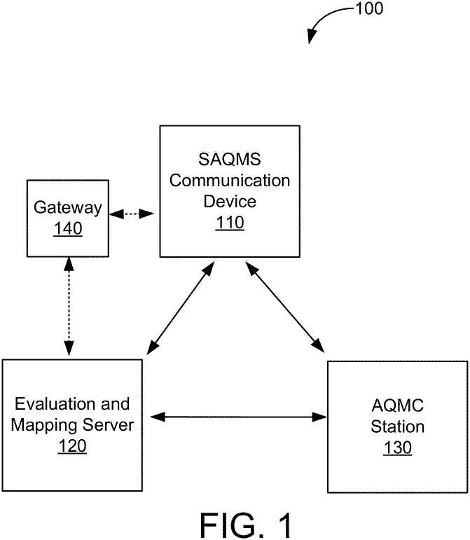

Referring now to FIG. 1, in various embodiments, an exemplary air quality measurement and communication system 100 includes portable SAQMS communication device(s) 110, a centralized evaluation and mapping server platform 120, and air quality measurement and calibration (AQMC) station 130. The evaluation and mapping server 120 is configured to acquire, map, and disseminate citizen-gathered air quality data provided from the portable SAQMS communication device(s) 110. Additionally, the evaluation and mapping server 120 maps high-resolution air quality data obtained from the sensors of the portable SAQMS communication device(s) 110. Prior to such mapping, these data are calibrated using AQMS sensor(s). In various embodiments, the AQMC sensors can be located and fixed in high-density locations around a geographic location, such as select spots having a high degree of pollutants.

In various embodiments, sensors integrated with the portable SAQMS communication device 110 may be at lower accuracy owing to smaller sized sensors than sensors integrated with the AQMC station 130. Accordingly, owing to size limitations, there may be instances when the accuracy tolerances of the sensors for the portable SAQMS communication device 110 may be lower than the sensors for the AQMC station 130 and, hence, the need for AQMC stations. Thus, personal sensor data provided from the portable SAQMS communication device 110 at or within a certain range of the geographic location (having a fixed AQMC station) can be compared to the high-resolution data provided from the AQMC station sensor 130 and used to determine an amount of data-drift or a level of resolution or accuracy experienced by the portable SAQMS communication device 110 and relayed to the evaluation and mapping server 120, such that the data acquired from the portable SAQMS communication device 110 can be calibrated by the evaluation and mapping server 120 to compensate for measurement inaccuracies and provide a higher reliability of data gathered and disseminated. In various embodiments, calibrated data can be derived from a plurality of locations having air quality data from the portable SAQMS communication device 110 and AQMC station(s) 130 at those locations.

In various embodiments, the portable SAQMS communication device 110 may communicate with one or more base stations, such as fixed AQMC stations 130 over a wide area network in real-time (e.g., low-power wide area network (WAN) system, such as LoRaWAN) or via a cellular network in which the fixed AQMC stations 130 can relay information to the evaluation and mapping server 120 using direct communications (e.g., cellular communication) or indirect communications (e.g., AQMC communications with the gateway device 140 over a WAN and communications between the gateway device 140 and the evaluation and mapping server 120 via the Internet, etc.).

In addition to, or in alternative to, some embodiments of the present disclosure utilize base stations in the form of a portable AQMC station 130 that can be housed in a user’s home, office, etc. Accordingly, the portable AQMC station 130 can have high-resolution air quality sensors whose measurements are relayed to the evaluation and mapping server 120 and used to determine measurement inaccuracies with low-resolution air quality data provided from a user’s portable SAQMS communication device 110 at the same location as the portable AQMC station 130. In one embodiment, the base station is driven by a circuit development board (e.g., ESP32 board) onto which the necessary sensors are integrated, such that the board is configured to provide data through a local microprocessor and web server (e.g., Arduino UNO and Bluehost web server). In various embodiments, the portable AQMC station 130 may be equipped with a global positioning system (GPS) sensor that can provide location data for the portable AQMC station 130. In the same manner, the portable SAQMS communication device 110 is also equipped with a GPS sensor such that it can provide location data for the portable SAQMS communication device 110 to the evaluation and mapping server 120.

In addition to the high-resolution sensors, the portable base station or AQMC station may also be equipped with battery charger circuitry that is configured to charge a battery of the portable SAQMS communication device. In various embodiments, the AQMC station may be equipped with a network adapter (e.g., WIFI adapter) to allow for the portable AQMC station to communicate with the evaluation and mapping server in real time. In other embodiments, the portable AQMC station may be equipped to communicate over a short-range channel (e.g., Bluetooth channel) with the user’s phone or tablet, such that the phone/tablet can relay data provided to/from the portable AQMC station from/to the evaluation and mapping server. In various embodiments, the upload/download of data at the portable AQMC station can be synchronized with mobile phone apps.

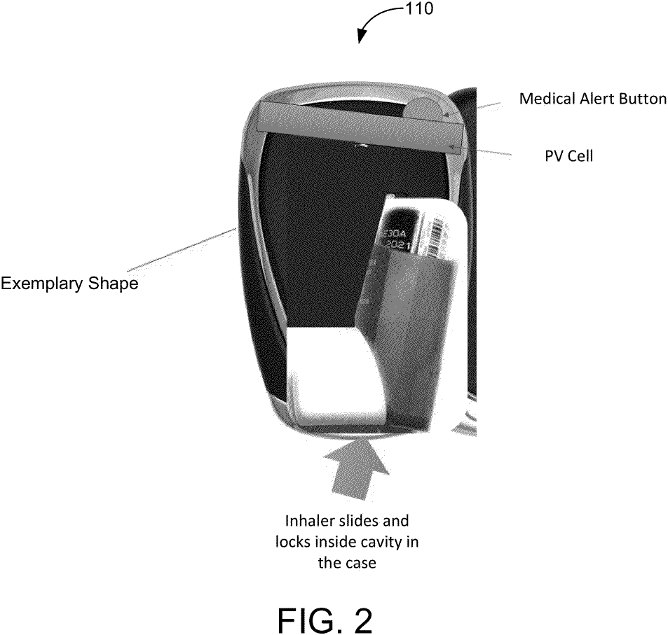

In various embodiments, an exemplary portable SAQMS communication device is in a form of a carrying case having a pouch in which an inhaler device can be inserted, stored, and protected from an accidental release of doses. In various embodiments, a shape of the case is adapted to fit a geometry of an asthma inhaler 300 (FIG. 2) and a size of inhaler cartridge 305 (FIG. 2) while also having sufficient area for integrating corresponding circuitry hardware. As such, in various embodiments, different shaped or sized asthma inhalers may utilize different versions of the portable air quality measurement communication device (e.g., SAQMS 110). An exemplary personal communication device includes a case opening to an inner pouch in which the inhaler device can be inserted (e.g., by sliding the inhaler device into the case). The carrying case can be attached to the belt or the individual’s garment using a lock system. Alternatively, in certain embodiments, an exemplary portable air quality measurement communication device may feature a pouch that can hold other item(s) besides an inhaler device or may be left empty. Accordingly, such a device can include a clip, hook, loop, etc. for attaching the portable air quality measurement communication device to a person’s clothing or accessory, such as a backpack, transport vehicle (e.g., bike), pet leash, etc. during a person’s travels that allows for collection of air quality data.

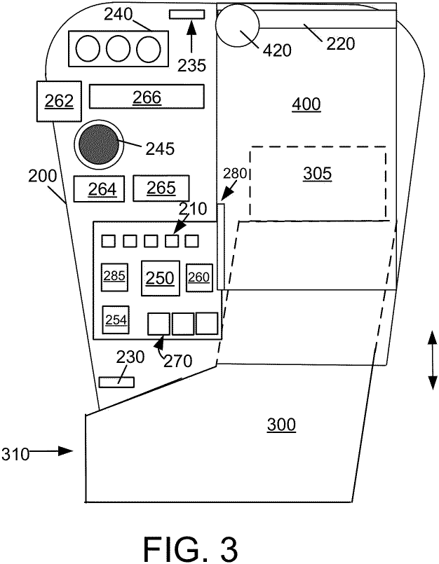

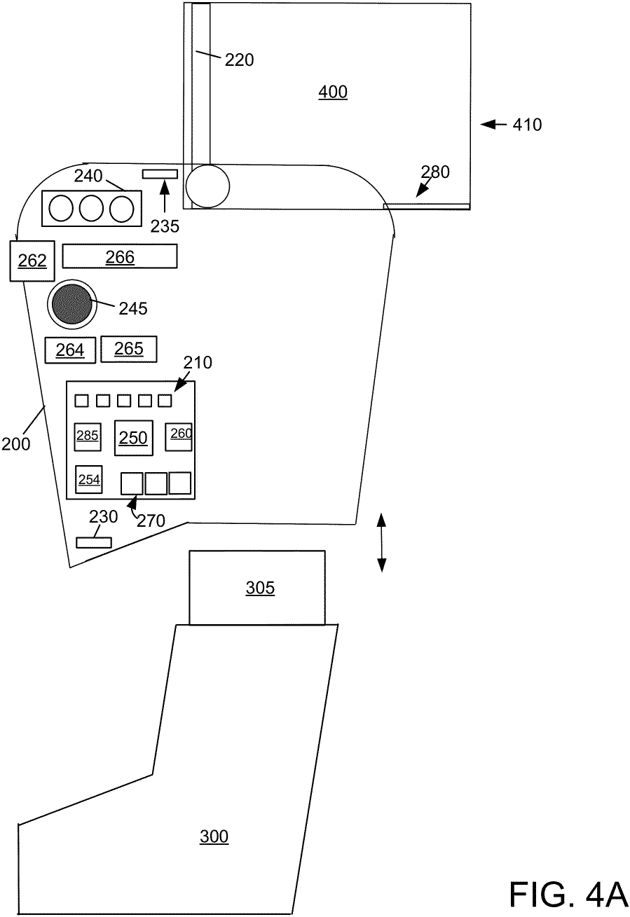

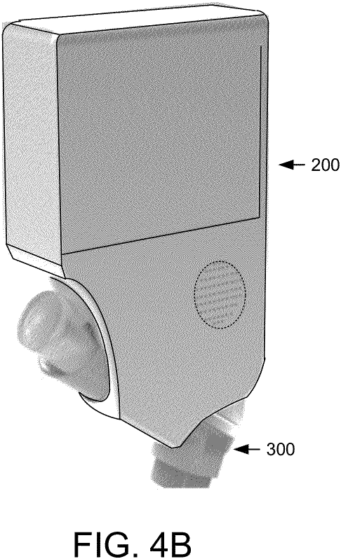

Referring back to the figures, FIG. 3 presents a computer-generated sketch of a case 200 of an exemplary SAQMS communication device 110 in conjunction with an asthma inhaler device 300. Additionally, FIG. 3-4A present computer-generated drawings of the case that correspond to FIG. 2 with an asthma inhaler device 300 inserted in the case (FIG. 3) and a spirometer device 400, a pulse oximeter 280, and the asthma inhaler device 300 not inserted in the case 200 (FIG. 4A), in accordance with various embodiments of the present disclosure. Correspondingly, FIG. 4B shows a prototype image of an exemplary case of the portable SAQMS communication device that corresponds to FIG. 2 with an asthma inhaler device inserted in the case. In various embodiments, the size of the case is approximately 4.5 inches × 2.5 inches which provides storage space for the required sensors and related circuitry. The table below (Table 1) provides exemplary sensor information for one non-limiting design:

TABLE 1

| | | | |
|---|---|---|---|
| Parameter | Item | Operating Conditions | Dimensions |
| NO₂, CO | MICS-4514 | Supply Voltage: 4.9 - 5 V Current Rating: 32/26 mA | 0.28 in × 0.2 in × 0.06 in |
| Temperature Humidity | BME/BMP280 | Supply Voltage: 3 V Current Rating: 9.9936 mA | 0.45 in x 0.6 in |
| PM_{2.5}, PM₁₀ | HPMA115CO-003 (Honeywell) | Supply Voltage Input: 5 V±0.2 V Supply Current: <80 mA (@ 25° C.±5° C.) | 1.73 in × 1.42 in × 0.48 in |
| Pulse Oximeter and Heart-Rate Sensor | Max30100 | Supply Voltage Input: 3 -5.5 V Supply Current: 0.6 - 1.2 mA | 0.5 in × 0.5 in |

In one embodiment, an individual removes the inhaler device 300 and rotates the spirometer device 400 along a pivot 420 attached to the case 200 in order to blow air into the same for lung measurements, as represented in FIG. 3-4A. In this case, a mouthpiece 410 of the spirometer tube may be extended. In alternative embodiments, an inner tube of the spirometer device may be pulled out from an outer tube in a telescopic manner.

In various embodiments, the pulse oximeter 280 is embedded to the inner tube of the spirometer, as shown in FIG. 3-4A. In this embodiment, the individual removes the inhaler and the spirometer tube is exposed. And, the individual places his/her index finger in the identified location inside the spirometer tube to obtain the readings. Alternatively, in various embodiments, the pulse oximeter 280 may be attached to a spring loaded tension cord and may be extended from a body of the SAQMS device (having an internal tension pulley coupled to the tension cord) (as opposed to be located in an inner tube of the spirometer 400).

In one embodiment, an opening to the pouch of the case 200 is on a bottom of the case 200. Integrated in the case 200 are a plurality of air quality sensors 210, such as two or more sensors for detecting temperature values, air levels of ozone (O₃), nitrogen oxide (NO₂), particulates (e.g., PM_{2.5}, PM₁₀, etc.), relative humidity (RH), in addition to global position data (e.g., GPS data), among others. Additionally, other controls and sensors may be integrated with the case 200, in various embodiments, including a pressure sensor 220 that can detect movement of air blown through a spirometer device 400, via a change in pressure. Alternatively, or in addition to, the inhaler device 300 may be removed from the case 200 and used outside of the case, as represented in FIG. 4A.

Further, in various embodiments, a spirometer device 400 having a mouthpiece 410 and a spirometer pressure sensor 220 can be positioned within a cavity of the case 200 or sandwiched between two halves of the case 200. Accordingly, the spirometer device 400 can be configured to measure a volume of air inspired and expired by the lungs of a user. As such, in various embodiments, a pressure sensor 220 is in fluidic communication with a tube of the spirometer device and is located at a middle portion or end portion of the spirometer tube. Alternatively, or in addition to, the spirometer device may be rotated away from the case 200 (via a pivot 420) and used outside of the case, as represented in FIG. 4A. To use the spirometer device 400, an individual can extend a mouthpiece portion of the spirometer inhaler/exhaler tube. The data from the pressure sensor is transmitted to the microcontroller unit 250 directly. In one embodiment, a pulse oximeter 280 located on the case (e.g., close to a mouth of the inhaler device) can be used by an individual. The data from this pulse oximeter device 280 is sent to the microcontroller unit 250.

In addition to monitoring air volume, the case is equipped with one or more microphones 230, 235 and circuitry inside or adjacent to the inhaler and/or spirometer mouthpiece(s) 310, 410 to record breathing sounds (e.g., labored breathing noises such as wheezing, crackling, stridor, etc. by a user) during use of the inhaler device 300 and/or spirometer device 400 that can be communicated to the evaluation and mapping server 120, where the server 120 can process the data and check the data against a database of breathing files to quantify a severity of the breathing recorded by the spirometer device 410, in various embodiments. The severity level of the breathing may cause a LED display 240 positioned on an outside of the case to be activated or caused to flash if the severity level is above a certain level, in some embodiments. Alternatively, or in addition to, the LED display 240 may display a certain color or pattern corresponding to the current breathing level. In some embodiments, the user may authorize for the alert data and/or breathing data to be transferred to a doctor’s office from the evaluation and mapping server 120. Also, the alert data and/or breathing data may be regularly deleted from the evaluation and mapping server 120 after their initial use and/or after sharing the data with an authorized medical personnel. In various embodiments, an alert button 245 on the case may be used by individual to send an alert signal to their family and friend circle based on their preferences. Alternatively, airflow data may be collectively gathered by citizens without disclosing any identifying personal information (i.e. personal identifier information) within a geographic location and supplied to the evaluation and mapping server 120 so that it may be used to quantify the air quality levels for the geographic location, e.g., real-time and/or future prediction of air quality based on artificial intelligence techniques. Accordingly, alerts can be published or indicated on air quality maps (e.g., by displaying certain buildings or areas in a certain color to indicate a particular air quality level) based on the collective usage data from individuals in a geographic region, such as air volume levels supplied by users of the spirometer/inhaler devices of exemplary portable SAQMS devices. Thus, in various embodiments, the mapping server can provide interactive access to post-processed data to view pollutant concentrations.

Connected to the sensors and other electronics of the exemplary multi-sensory SAQMS communication device 110 is a microcontroller unit 250 housed or integrated with the portable SAQMS communication device. As a non-limiting example, the plurality of air quality sensors 210, GPS sensor 260, pressure sensor(s) 220, and pulse oximeter sensor 285 may be situated in the physical proximity of the microcontroller unit 250. Depending on a density of high quality or high resolution air quality sensors via fixed AQMC stations 130 in a geographic area, a sampling or sensing frequency of the air quality sensors at the portable SAQMS communication device 110 can be adapted or changed by the microcontroller unit 250.

The microcontroller unit 250 communicates with the sensors 210, a power management system (including, for example, a battery 265, a charging port 262, charging circuitry 264, a photovoltaic (PV) cell 266 (positioned on an outside of the case), etc.), a LED display interface system 240, a data storage 254, and a wireless communication components 270 (e.g., cellular circuitry, WIFI circuitry, Bluetooth circuitry, WAN circuitry, etc.). The wireless communication system transfers the data from the sensors or storage systems to a mobile application, evaluation and mapping server, base stations, and/or other devices through the microcontroller unit using one or more wireless communication protocols, such as cellular, WIFI, Bluetooth, LoRaWAN, etc.

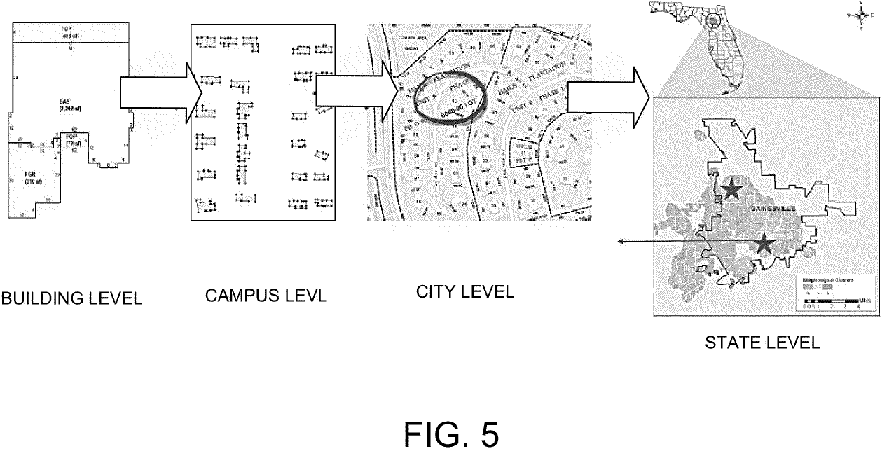

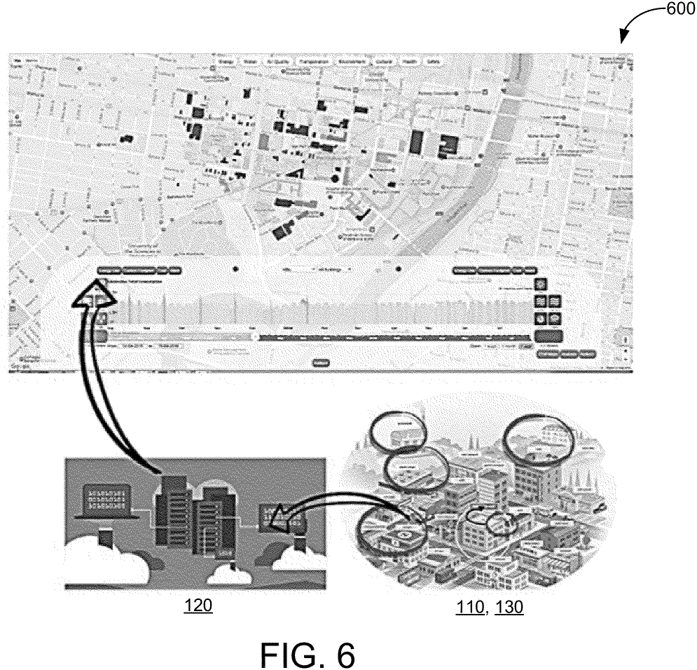

Referring now to the evaluation and mapping server 130, the server is configured to collect data, analyze data and settings, generate a map of environmental data (e.g., a real-time map of air quality data) for geographic areas, and publish and/or distribute the map data for consumption by interested parties (e.g., users of inhaler devices, third parties, the public, etc.). In various embodiments, such maps can reliably and accurately depict air quality data using low-cost and/or low-energy sensors (very small in size and lightweight such that they can be easily transported by subjects), yet generate high-resolution, high-quality, calibrated data in real-time. This data can also be used by other existing providers (third-party vendors) to facilitate appropriate warnings to their customers or subjects (e.g., patients). Collection of data by the evaluation and mapping server 120 comprises citizen-gathered data from citizens of the public in collaboration with the evaluation and mapping server 120, in various embodiments. As such, the evaluation and mapping sever 120 can track a crowd of users’ environmental surroundings and/or the users’ usage data in view of the environmental surroundings. Data used for generating maps can be populated based on statistical analysis/interpolation of the citizen-gathered data. Advantageously and in accordance with embodiments of the present disclosure, air quality data can be acquired inside and outside of buildings for real-time mapping/monitoring and alerts can be provided to the public at large or air quality data/maps can be supplied to third parties who can alert their respective customers/subjects, as a non-limiting example. For example, varying scales or levels of the map may be available, such as a building, campus, city, and state scales/levels of the map, as represented in FIG. 5. Thus, at the various levels or scale, the map can identify regions (e.g., a building at a campus view, etc.) that are considered as a safe zone and/or a “danger zone” with respect to having a high level of pollutants by indicating these regions in a certain color (e.g., red), with a certain indicator or icon, etc., as represented in the map shown on the top left portion of FIG. 6. Likewise, safe zones could be displayed using a different color (e.g., green), in some embodiments.

In one embodiment, an exemplary method of evaluating and mapping environmental data comprises receiving air quality sensor data from a plurality of portable SAQMS communication devices 110 from a plurality of citizens in a geographic location; receiving air quality sensor data from an air quality measurement and calibration (AQMC) station 130 in the geographic location; determining a level of resolution for one or more sensors of the portable SAQMS communication device 110 at a central evaluation and measurement service 130; correcting air quality data received from the portable SAQMS communication device 110 to compensate for the determined level of inaccuracy at the central evaluation and measurement service 130; and generating a map 600 (FIG. 6) of air quality data based on the corrected air quality data of the portable SAQMS communication device 110 and a plurality of other portable SAQMS communication devices 110. The foregoing process is illustrated in FIG. 6 by the depiction of air quality sensor data being supplied by portable SAQMS communication devices 110 and air quality measurement and calibration stations 130 to the central evaluation and measurement service 130, in which the data is corrected, calibrated, and combined with other sensor data from other portable SAQMS communication devices 110 to generate an air quality map 600.

In certain embodiments, to obtain real-time data from SAQMS communication device(s) 110, the devices can be programmed to switch its sensors on/off (frequency) based on the plurality of SAQMS devices 110 in a specific geographic area. In this way, an individual SAQMS 110 does necessarily have to keep all of its sensors activated (which will drain its battery). Rather, in various embodiments, if there are numerous citizens with SAQMS devices 110 within a geographic area, a control center, such as evaluation and mapping server 120, can command for a sensor sampling frequency of those citizens’ SAQMS devices 110 to be staggered. Thus, real-time data may be intelligently obtained without unnecessarily draining an individual SAQMS battery.

In one embodiment, an exemplary method of acquiring air quality data from a portable SAQMS communication device 110 comprises measuring a plurality of air quality levels of ambient air at a particular location at a portable SAQMS communication device 110 and sending the air quality levels and current location data to a base station, which can include an evaluation and mapping server 120. In various embodiments, the air quality levels include one or more of O₃, PM_{2.5}, PM₁₀, NO₂, temperature, and RH (relative humidity) levels or values. In various embodiments, the method further includes recording a file of breathing sounds by a user of an inhaler device 300 recorded by microphone 230 integrated with the portable air quality measurement communication device and sending the sound file to the evaluation and mapping server 120 with current location data.

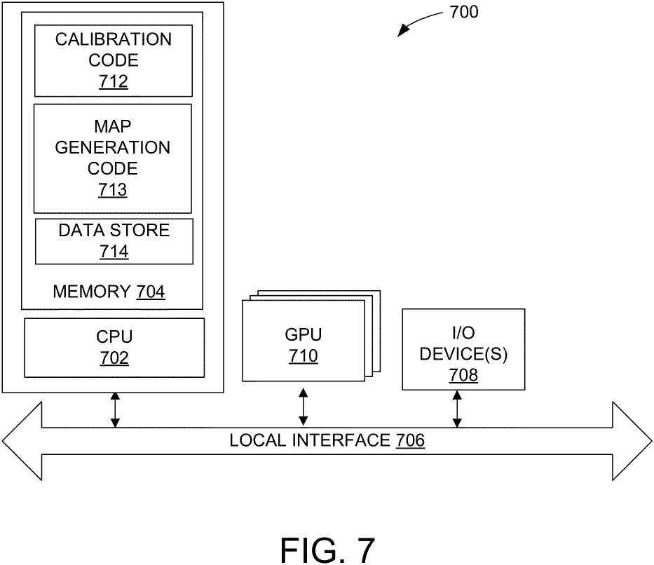

FIG. 7 depicts a schematic block diagram of a computing device 700 that can be used to implement various embodiments of the present disclosure, such as the evaluation and mapping server 120. An exemplary computing device 700 includes at least one processor circuit, for example, having a processor 702 and a memory 704, both of which are coupled to a local interface 706, and one or more input and output (I/O) devices 708. The local interface 706 may comprise, for example, a data bus with an accompanying address/control bus or other bus structure as can be appreciated. The computing device 700 may further include Graphical Processing Unit(s) (GPU) 710 that are coupled to the local interface 706 and may utilize memory 704 and/or may have its own dedicated memory. The CPU and/or GPU(s) can perform various operations such as image enhancement, graphics rendering, image/video processing, and any of the various operations described herein.

Stored in the memory 704 are both data and several components that are executable by the processor 702. In particular, stored in the memory 704 and executable by the processor 702 are code for correcting or calibrating air quality sensor data 712 and generating air quality maps 713. Also stored in the memory 704 may be a data store 714 and other data. In addition, an operating system may be stored in the memory 704 and executable by the processor 702. The I/O devices 708 may include input devices, for example but not limited to, a keyboard, mouse, etc. Furthermore, the I/O devices 708 may also include output devices, for example but not limited to, a printer, display, etc.

Certain embodiments of the present disclosure can be implemented in hardware, software, firmware, or a combination thereof. If implemented in software, the air quality sensor calibration and/or map generation logic or functionality are implemented in software or firmware that is stored in a memory and that is executed by a suitable instruction execution system. If implemented in hardware, the calibration and/or map generation logic or functionality can be implemented with any or a combination of the following technologies, which are all well known in the art: discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

It should be emphasized that the above-described embodiments are merely possible examples of implementations, merely set forth for a clear understanding of the principles of the present disclosure. Many variations and modifications may be made to the above-described embodiment(s) without departing substantially from the principles of the present disclosure. All such modifications and variations are intended to be included herein within the scope of this disclosure.

1. A method of evaluating air quality sensor data comprising:
receiving air quality sensor data from a plurality of portable smart air quality measurement system (SAQMS) communication devices in a form of an asthma inhaler carrying case from a plurality of citizens in a geographic location;
receiving air quality sensor data from an air quality measurement and calibration (AQMC) station in the geographic location;
determining a level of resolution for one or more sensors of a portable SAQMS communication device at a central evaluation and measurement service;
correcting air quality data received from the portable SAQMS communication device to compensate for the determined level of resolution at the central evaluation and measurement service; and
generating a map of air quality data based on at least the corrected air quality data of the portable SAQMS communication device and a plurality of other portable SAQMS communication devices.

2. The method of claim 1, further comprising attaching the portable SAQMS communication device to an asthma inhaler.

3. The method of claim 2, wherein the portable SAQMS communication device is integrated with a spirometer device, the spirometer device having a pressure sensor and a spirometer tube coupled to the pressure sensor, the method further comprising extending a mouthpiece of the spirometer device away from a body of the portable SAQMS communication device.

4. The method of claim 3, wherein the portable SAQMS communication device is integrated with a pulse oximeter device.

5. The method of claim 4, wherein the pulse oximeter device is located in an inner tube of the spirometer device.

6. The method of claim 1, wherein the portable SAQMS communication device comprises a plurality of air quality sensors, the plurality of air quality sensors including sensors for measuring O₃, PM_{2.5}, NO₂, temperature, and relative humidity levels.

7. The method of claim 1, further comprising publishing the generated map for public consumption.

8. The method of claim 1, wherein the portable SAQMS communication device communicates with one or more servers for the central evaluation and measurement service using cellular communications.

9. The method of claim 1, wherein the portable SAQMS communication device communicates with one or more servers for the central evaluation and measurement service using WiFi communications.

10. The method of claim 1, wherein the portable SAQMS communication device communicates with the air quality measurement and calibration (AQMC) station using short range communications.

11. The method of claim 1, wherein the generated map indicates an air quality level inside a building depicted in the generated map.

12. The method of claim 1, wherein the generated map identifies regions having a high level of pollutants and safe zones.

13. The method of claim 1, further comprising receiving usage data from the plurality of portable SAQMS communication devices, wherein the usage data indicates a volume of air inspired and expired by the lungs of a plurality of users during use of asthma inhaler devices.

14. The method of claim 1, further comprising receiving usage data from the plurality of portable SAQMS communication devices, wherein the usage data indicates occurrences when respective asthma inhaler devices are used by a plurality of users.

15. The method of claim 1, wherein the air quality sensor data is provided with location data for the geographic location.

16. The method of claim 1, further comprising receiving breathing sound data from the plurality of portable SAQMS communication devices, wherein the breathing sound data are recorded during usage of a spirometer device or an asthma inhaler that is coupled or attached to a respective portable SAQMS communication device.

17. A system of evaluating air quality sensor data comprising:
a carrying case having an interior pouch;
a spirometer device integrated with the carrying case, wherein the spirometer device has a pressure sensor and a spirometer tube coupled to the pressure sensor, wherein the spirometer devices a mouthpiece that is configured to be extended away from a body of the carrying case;
a plurality of air quality sensors integrated with the carrying case, the plurality of air quality sensors including sensors for measuring O₃, PM_{2.5}, NO₂, temperature, and relative humidity levels;
a global positioning system (GPS) sensor integrated with the carrying case and configured to provide location data for the carrying case; and
hardware circuitry configured to transmit sensor data from at least the pressure sensor, air quality sensors, or GPS sensor to a base station.

18. The system of claim 17, wherein the interior pouch is adapted to receive an asthma inhaler device.

19. The system of claim 17, further comprising one or more microphones adjacent to the mouthpiece of the spirometer, wherein the sensor data transmitted by the hardware circuitry further comprises breathing sounds recorded via the one or more microphones.

20. The system of claim 17, further comprising a pulse oximeter device integrated with the carrying case, wherein the pulse oximeter device is located in an inner tube of the spirometer device, wherein the sensor data transmitted by the hardware circuitry further comprises data obtained by the pulse oximeter device.
